(19) United States
(12) Patent Application Publication
(10) Pub. No.: US 2021/0371808 A1
(43) Pub. Date: Dec. 2, 2021

(54) ACHROMOBACTER XYLOSOXIDANS WITH MONOMETHYLAMINE DEGRADABILITY AND APPLICATION THEREOF

(71) Applicant: Guangdong University of Technology, Guangzhou (CN)

(72) Inventors: Guiying LI, Guangzhou (CN); Shiai LI, Guangzhou (CN); Taicheng AN, Guangzhou (CN); Hongli LIU, Guangzhou (CN); Jiangyao CHEN, Guangzhou (CN)

(21) Appl. No.: 16/758,989

(22) PCT Filed: Mar. 20, 2018
(86) PCT No.: PCT/CN2018/079532

§ 371 Date: Apr. 24, 2020

(30) Foreign Application Priority Data
Nov. 7, 2017 (CN) 201711084294.0

(51) Int. Cl. C12N 1/20

(52) U.S. Cl. CPC C12N 1/205; C12R 2001/025

(57) ABSTRACT

The present invention discloses a strain of Achromobacter xylosoxidans with monomethylamine degradability and the application thereof. This strain, named Achromobacter xylosoxidans GDUTAN5, was deposited on May 24, 2017 in the China Center for Type Culture Collection in Wuhan University (No. 299 Bayi Road, Wuchang District, Wuhan City, Hubei Province) with a deposit number of CCTCC NO: M 2017285. This Achromobacter xylosoxidans GDUTAN5 was Gram-negative and rod-shaped, and the colony appeared to be round, light yellow, opaque and smooth, having a diameter of 1-2 mm. The Achromobacter xylosoxidans GDUTAN5 of the present invention can be applied to environmental restoration, degrading monomethylamine in the environment at a high degradation efficiency. When it degraded monomethylamine for 96 h at a substrate concentration of 5 mg/L, the degradation efficiency could reach 92.3%.

This application claims the priority of Chinese Patent Application No. 201711084294.0 that is entitled “Achromobacter Xylosoxidans with Monomethylamine Degradability and Application Thereof” and was submitted to the China Patent Office on Nov. 7, 2017. The entire contents thereof are incorporated herein by reference.

### FIELD OF THE INVENTION

The present invention belongs to the field of microbial technology, and more particularly relates to a strain of Achromobacter xylosoxidans with monomethylamine degradability and the application thereof.

### BACKGROUND OF THE INVENTION

Methylamine organic compounds, such as monomethylamine, dimethylamine and trimethylamine, are the main raw materials or intermediates used in the pharmaceutical or chemical industries for the production of dyes, rubbers, pesticides, pharmaceuticals, cosmetics, corrosion inhibitors, and so on. They not only have a strong odor, but also cause strong irritation to the skin, eyes, nose, throat or respiratory system of humans exposed to such contaminants. Among them, monomethylamine is the simplest methylamine compound and widely used in industry. However, due to its low boiling point and high vapor pressure, it evaporates into the environment in large quantities during production and use, endangering the human health and natural environment. In addition, monomethylamine has an extremely low olfactory threshold and a strong fish odor at low concentrations, which also adversely affects human physiological and mental health. Therefore, it is very necessary and urgent to effectively remove monomethylamine from the environment. The use of microorganisms to degrade organic pollutants in the environment is a very environmentally friendly method. One of the most studied monomethylamine-degrading bacteria is Methylobacterium AM1, which is widely found in water, soil and air and capable of using monomethylamine as the sole source of carbon and nitrogen. It has also been found that the marine bacterium Roseobacter is capable of using monomethylamine, dimethylamine and trimethylamine as important nitrogen sources and possible energy sources. However, literature reports on the degradation of monomethylamine using a new single strain isolated from landfill leachate are very limited. Therefore, screening for low-cost, high-efficiency monomethylamine-degrading bacteria is of great significance for the purification of malodorous organic nitrogen-containing exhaust gas. The Achromobacter xylosoxidans is a rod-shaped Gram-negative bacterium, and can form white, round, irregularly edged, rough colonies on the surface of a solid medium. So far, no relevant reports and patents on the degradation of monomethylamine using Achromobacter xylosoxidans have been seen.

### CONTENTS OF THE INVENTION

An object of the present invention is to provide a strain of Achromobacter xylosoxidans with monomethylamine degradability to overcome the deficiencies of the prior art. Achromobacter xylosoxidans GDUTAN5, belonging to a new variant of the Achromobacter xylosoxidans genus, has excellent monomethylamine degradability, and can degrade the monomethylamine in the environment at a high degradation efficiency.

Another object of the present invention is to provide an application of the above-described Achromobacter xylosoxidans with monomethylamine degradability in environmental restoration.

The objects of the present invention are achieved by the following technical solution:

The present invention, in a first aspect, provides a strain of Achromobacter xylosoxidans with monomethylamine degradability named Achromobacter xylosoxidans GDUTAN5, which was deposited on May 24, 2017 in the China Center for Type Culture Collection in Wuhan University, Wuhan City, Hubei Province with a deposit number of CCTCC NO: M 2017285.

The morphological characteristics of the Achromobacter xylosoxidans with monomethylamine degradability of the present invention are as follows:

(a) By using the physiological and biochemical identification methods of bacteria and electron microscopy, it was revealed that the Achromobacter xylosoxidans screened out was Gram-negative and rod-shaped with cell staining.

(b) Morphological characteristics of the colonies: After 24 h of culture in an LB solid medium, the colony appeared to be round, light yellow, opaque and smooth, with a diameter of 1-2 mm.

The main physiological and biochemical characteristics of the Achromobacter xylosoxidans with monomethylamine degradability of the present invention are shown in Table 1 below:

TABLE 1
Main physiological and biochemical characteristics
of Achromobacter xylosoxidans

| Items | Test results |
|---|---|
| Arabinose | − |
| Xylose | − |
| Glucose | − |
| Mannitol | − |
| Citrate utilization | + |
| DNA hydrolysis | − |
| V-P test | − |
| Nitrate (reduction) | − |
| Starch hydrolysis | − |
| Gelatin liquefaction | − |
| Anaerobic growth | + |
| 2% NaCl growth | + |
| 5% NaCl growth | + |
| pH = 5.5 growth | + |
| pH = 9.0 growth | + |
| Gram staining | − |
| 50° C. growth | − |
| 15° C. growth | + |

The 16S rDNA sequence of the Achromobacter xylosoxidans with monomethylamine degradability of the present invention is set forth in SEQ ID NO: 1.

By alignment analysis of the 16S rDNA sequence, it was found that the homology between the strain of the present invention and Achromobacter xylosoxidans GAD3 reached 67%. By combining the morphological characteristics, growth conditions, and physiological and biochemical identification results of the bacteria, it was determined that Achromobacter xylosoxidans GDUTAN5 belongs to a new variant of the Achromobacter xylosoxidans genus and was so named.

The present invention, in a second aspect, provides an application of the Achromobacter xylosoxidans with monomethylamine degradability in environmental restoration.

The Achromobacter xylosoxidans with monomethylamine degradability of the present invention is capable of degrading monomethylamine in the environment when used in environmental restoration.

Further, the environment includes atmosphere, water or soil.

Compared with the prior art, the present invention has the following beneficial effects:

1. The strain of Achromobacter xylosoxidans GDUTAN5 of the present invention was screened for the first time out of the landfill leachate of a landfill in Guangzhou City, Guangdong Province, having monomethylamine degradability.

2. The Achromobacter xylosoxidans of the present invention has the ability to degrade monomethylamine; when it degraded monomethylamine for 96 h at a substrate concentration of 5 mg/L, the degradation efficiency could reach 92.3%.

### BRIEF DESCRIPTION OF THE DRAWINGS

FIG. 1 shows the morphology of Achromobacter xylosoxidans GDUTAN5 of the present invention under an electron microscope.

### DETAILED DESCRIPTION OF THE EMBODIMENTS

The present invention will be further described with reference to the following specific examples, but these examples should not be construed as limiting the present invention. Unless otherwise specified, the technical means used in the examples are conventional means well known to those skilled in the art. Unless otherwise indicated, the reagents, methods and devices employed in the present invention are routine in the art.

### EXAMPLE 1

A strain of Achromobacter xylosoxidans with monomethylamine degradability, named Achromobacter xylosoxidans GDUTAN5, was deposited on May 24, 2017 in the China Center for Type Culture Collection in Wuhan University, Wuhan City, Hubei Province with a deposit number of CCTCC NO: M 2017285.

The Achromobacter xylosoxidans GDUTAN5 of the present example was isolated and screened from leachate of a landfill in Guangzhou City, Guangdong Province. The isolation and purification methods were as follows: The acclimation medium used was an inorganic salt culture aqueous solution (each 1000 mL of the inorganic salt culture aqueous solution contained K₂HPO₄.3H₂O 1.2 g/L, KH₂PO₄ 1.2 g/L, NH₄Cl 0.4 g/L, MgSO₄.7H₂O 0.2 g/L, FeSO₄.7H₂O 0.01 g/L, and 1 mL of a trace element aqueous solution, wherein the trace element aqueous solution contained CaCl₂.2H₂O 0.2 g/L. MnSO₄.4H₂O 0.2 g/L. CuSO₄.2H₂O 0.01 g/L, ZnSO₄.7H₂O 0.2 g/L, CoCl₂.6H₂O 0.09 g/L, Na₂MoO₄.2H₂O 0.12 g/L, and H₃BO₃ 0.006 g/L). First, 1 mL of the landfill leachate was taken and diluted 100 times, inoculated into a nutrient broth, and aerobically cultured at 37° C. for 1 day in a shaker at a rotational speed of 150 rpm. 1 mL of the enriched bacterial solution was taken and inoculated in an inorganic salt nutrient solution containing monomethylamine, and aerobically cultured at 37° C. for 5 days in a shaker at a rotational speed of 150 rpm, and then moved to the next concentration in an inoculating amount of 10% for acclimation, with the substrate acclimation gradients respectively at 10, 20, 50 and 100 mg/L. The final-concentration acclimation solution was diluted by 10⁻¹ to 10⁻⁶, 200 μL of the dilutions diluted by 10⁻⁵ and 10⁻⁶ was respectively applied to a solid agar plate with monomethylamine as the sole carbon source (the solid medium containing monomethylamine was obtained by adding 18 g of agar powder and 4 mg of monomethylamine to per liter of the above inorganic salt medium), and the plates were placed into an incubator to culture at 35° C. for 3 days; a single colony of different morphology was selected for determination of the degradation efficiency of monomethylamine, and the strain with the highest degradation efficiency was selected for purification.

Determination of degradation efficiency: Sampling periodically during the biodegradation of monomethylamine, and determining the degradation efficiency spectrophotometrically. Degradation efficiency=(initial concentration−final concentration/initial concentration.

Spectrophotometric determination of monomethylamine concentration: Taking a certain amount of the monomethylamine degradation solution into a 10 mL colorimetric tube, diluting to 2.0 ml. with an absorption solution (0.01 mol/L HCI), and respectively adding 4.0 mL of a buffer (obtained by dissolving 4.08 g of potassium dihydrogen phosphate and 1.6 g of borax in 80 mL of distilled water, adding 6.35 mL of 5.0 mol/L NaOH solution, and diluting to 100 mL with water) and 0.4 mL of diazonium salt solution (obtained by adding 1.0 mL of sodium nitrite solution to 10 mL of p-nitrophenylamine hydrochloride solution and mixing), shaking well, letting stand for 40 min, adding 1.0 mL of 5 mol/L NaOH solution, mixing, letting stand for 20 min, and performing colorimetric quantification at 510 nm.

The purified colonies were identified, with the results as follows:

(1) Morphological characteristics of the bacteria:

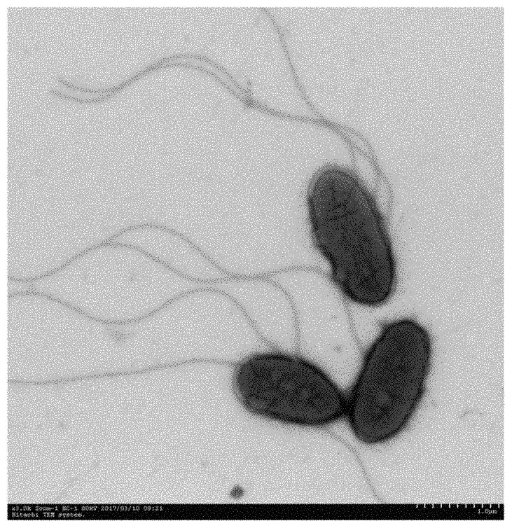

a. By using the conventional physiological and biochemical identification methods of bacteria and electron microscopy, it was revealed that the Achromobacter xylosoxidans screened out was Gram-negative with cell staining; under the electron microscope, the bacterium was rod-shaped with flagella around, and had a size of (0.6 to 1.0) μm×(1.3 to 1.8) μm, as shown in FIG. 1;

b. morphological characteristics of the colonies: after 24 h of culture in an LB solid medium, die colony appeared to be neatly edged, round, light yellow and opaque, having a diameter of 1-2 mm; and

c. the main physiological and biochemical characteristics of Achromobacter xylosoxidans are shown in Table 2.

TABLE 2
Physiological and biochemical characteristics
of Achromobacter xylosoxidans

| Items | Test results |
|---|---|
| Arabinose | − |
| Xylose | − |
| Glucose | − |
| Mannitol | − |
| Citrate utilization | + |
| DNA hydrolysis | − |
| V-P test | − |
| Nitrate (reduction) | + |
| Starch hydrolysis | − |
| Gelatin liquefaction | − |
| Anaerobic growth | + |
| 2% NaCl growth | + |
| 5% NaCl growth | + |
| pH = 5.5 growth | + |
| pH = 9.0 growth | + |
| Gram staining | − |
| 50° C. growth | − |
| 15° C. growth | + |

The above results indicate that the selected bacteria of the present invention had physiological and biochemical characteristics very similar to those of the Achromobacter xylosoxidans genus.

(2) Extracting bacterial genomic DNA and using bacterial 16S rDNA universal primers:

Upstream primer:
F27 (5′-AGTTTGATCMTGGCTCAG-3′)
Downstream primer:
R1492 (5′-GGTTACCTTGTTACGACTT-3′)

The entire 16S rDNA gene was amplified, with the sequencing results as shown in SEQ ID NO: 1.

By aligning the 16S rRNA gene sequence of 1359 bp in length as shown in SEQ ID NO: 1 with the gene sequence registered in the Genbank, it was found that the homology between the strain and Achromobacter xylosoxidans GAD3 was up to 67%.

Based on the above physiological and biochemical characteristics and 16S rRNA gene sequencing results, the selected strain of the present invention should belong to a new variant of the Achromobacter xylosoxidans genus, and was named Achromobacter xylosoxidans GDUTAN5.

The Achromobacter xylosoxidans GDUTAN5 was deposited on May 24, 2017 in the China Center for Type Culture Collection (CCTCC) in Wuhan University (No. 299 Bayi Road, Wuchang District, Wuhan City, Hubei Province) with a deposit number of CCTCC NO: M 2017285.

### EXAMPLE 2

In this example, Achromobacter xylosoxidans GDUTAN5 was applied to environmental restoration, and could degrade monomethylamine in the environment. The environment includes atmosphere, water or soil.

The monomethylamine degradability of the selected Achromobacter xylosoxidans GDUTAN5 of the present invention was tested as follows:

The inorganic salt medium was prepared according to the needs of the degradation experiment: Adding 100 mL of an inorganic salt solution to a 300 mL serum bottle (per 100 mL of the inorganic salt aqueous solution contains K₂HPO₄.3H₂O 0.12 g, KH₂PO₄ 0.12 g, NH₄Cl 0.04 g, MgSO₄.7H₂O 0.02 g, FeSO₄.7H₂O 0.001 g. CaCl₂.2H₂O 0.02 g, MnSO₄.4H₂O 0.02 g, CuS)₄.2H₂O 0.001 g, ZnSO₄.7H₂O 0.02 g, CoCl₂.6H₂O 0.009 g, Na₂MoO₄.2H₂O 0.012 g, H₃BO₃ 0.0006 g, and double distilled water 100 mL), and autoclaving at 121° C. for 30 min. First, activating the selected Achromobacter xylosoxidans GDUTAN5 with monomethylamine degradability in a nutrient broth medium (beef cream 3.0 g/L, peptone 10.0 g/L, and NaCl 5.0 g/L) at 30° C. for 24 h in a shaker at 100 rpm; and then, centrifuging the bacterial solution, collecting the bacteria, washing three times with a phosphate buffer, resuspending in 10 mL of an inorganic salt solution, and inoculating 1.0 mL of the bacterial solution into 100 mL of the inorganic salt solution containing different concentrations of monomethylamine; wherein the concentration of monomethylamine was 5, 10, 40, 70, 100 and 130 mg/L, respectively, the pH of the inorganic salt was 7, the reaction was carried out at 30° C. for 96 h in the shaker at 100 rpm, and sampling was made periodically to determine the degradation efficiency spectrophotometrically. The degradation efficiency was measured in the same way as in Example 1, and the results are shown in Table 3.

TABLE 3
Degradation efficiencies of different initial concentrations of
monomethylamine degraded by Achromobacter xytosoxidans GDUTAN5

| Monomethylamine | Degradation |
|---|---|
| concentration (mg/L) | efficiency |
| 5 | 92.3% |
| 10 | 84.1% |
| 40 | 65.5% |
| 70 | 49.3% |
| 100 | 35.1% |
| 130 | 20.6% |

As can be seen from Table 3, the Achromobacter xylosoxidans GDUTAN5 screened out by the present invention could degrade monomethylamine under this condition up to 92.3%.

The above examples are preferred embodiments of the present invention, but the embodiments of the present invention are not limited thereto, and any other alterations, modifications, substitutions, combinations, and simplifications made without departing from the spirit and principle of the present invention should all be equivalent replacements and included in the scope of protection of the present invention.

1. A strain of Achromobacter xylosoxidans with monomethylamine degradability, characterized in that: this strain, named Achromobacter xylosoxidans GDUTAN5, was deposited on May 24, 2017 in the China Center for Type Culture Collection in Wuhan University, Wuhan City, Hubei Province with a deposit number of CCTCC NO: M 2017285.

2. The Achromobacter xylosoxidans with monomethylamine degradability according to claim 1, characterized in that: the 16S rDNA sequence of the Achromobacter xylosoxidans GDUTAN5 is set forth in SEQ ID NO: 1.

3. An application of the Achromobacter xylosoxidans with monomethylamine degradability according to claim 1 in environmental restoration.

4. The application of the Achromobacter xylosoxidans with monomethylamine degradability in environmental restoration according to claim 3, characterized in that: the Achromobacter xylosoxidans GDUTAN5 is capable of degrading monomethylamine in the environment.

5. The application of the Achromobacter xylosoxidans with monomethylamine degradability in environmental restoration according to claim 3, characterized in that: the environment includes atmosphere, water or soil.


SEQUENCE LISTING (1 sequence)

SEQ ID NO: 1 - DNA, 1359 bp, Achromobacter xylosoxidans
caagtcgaacggcagcacggacttcggtctggtggcgagtggcgaacgggtgagtaatgtatcggaacgtgcccagtagcgggggataactacgcgaaagcgtagctaataccgcatacgccctacgggggaaagcaggggatcgcaagaccttgcactattggagcggccgatatcggattagctagttggtggggtaacggctcaccaaggcgacgatccgtagctggtttgagaggacgaccagccacactgggactgagacacggcccagactcctacgggaggcagcagtggggaattttggacaatgggggaaaccctgatccagccatcccgcgtgtgcgatgaaggccttcgggttgtaaagcacttttggcaggaaagaaacgtcgcgggttaataccccgcgaaactgacggtacctgcagaataagcaccggctaactacgtgccagcagccgcggtaatacgtagggtgcaagcgttaatcggaattactgggcgtaaagcgtgcgcaggcggttcggaaagaaagatgtgaaatcccagagcttaactttggaactgcatttttaactaccgggctagagtgtgtcagagggaggtggaattccgcgtgtagcagtgaaatgcgtagatatgcggaggaacaccgatggcgaaggcagcctcctgggataacactgacgctcatgcacgaaagcgtggggagcaaacaggattagataccctggtagtccacgccctaaacgatgtcaactagctgttggggccttcgggccttggtagcgcagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtctggaatgccgaagagatttggcagtgctcgcaagagaaccggaacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcgggacagagggtcgccaacccgcgagggggagccaatcccagaaacccgatcgtagtccggatcgcagtctgcaactcgactgcgtgaagtcggaatcgctagtaatcgcggatcagcatgtcgcggtgaatacgttcccgggtcttgtacacaccgcccgtcacaccatgggagtgggtttt